(19) United States
(12) Patent Application Publication
(10) Pub. No.: US 2015/0272836 A1
(43) Pub. Date: Oct. 1, 2015

(54) TOTAL BODY BABY WIPE

(71) Applicant: JOHNSON & JOHNSON CONSUMER COMPANIES, INC., Skillman, NJ (US)

(72) Inventors: Patricia Bonner, Branchburg, NJ (US); Belinda Mary Driscoll, London (GB); John F. Poccia, III, Monmouth Beach, NJ (US); Nathalie Quiry, Hondouville (FR)

(21) Appl. No.: 14/230,500

(22) Filed: Mar. 31, 2014

(51) Int. Cl. A61K 8/02; A61Q 19/10; D04H 1/425; D04H 1/492; D04H 1/435

(52) U.S. Cl. CPC A61K 8/0208; D04H 1/492; D04H 1/435; D04H 1/425; A61Q 19/10; D10B 2201/24; D10B 2331/04; D10B 2509/00

(57) ABSTRACT

The invention relates to a wet wipe product comprising a substrate and an emulsion. The wet wipe is sized and configured to enable a user to effectively cleanse an infant's entire body with a single wipe. The wipes are preferably made from nonwoven fabrics. The emulsion is particularly formulated to provide a mild cleansing with little or no irritation or drying to an infant's skin, hair or eyes.

### FIELD OF THE INVENTION

Wipe products have become an important product category that has found a wide variety of applications for adults and babies. Common examples of wipe products include face or body cleansing wipes, wipes for skin treatment, and skin conditioning wipes. Over the last couple of decades wet wipes have become successful as products particularly suited for these applications. These products typically are manufactured by impregnating sheets made of non-woven fabric with a suitable liquid, the formulation of which can vary widely, depending on the intended end use of the wipe.

Recent innovations in the wipes area included improvements in the fabric, in the impregnating liquid as well as in product packaging. Initially, wet wipe products were made of traditional non-woven materials based on paper making technology (pulp based products). These products were well accepted but were often deficient in softness of the fabric material. With the introduction of spunlace non-woven technology, products having superior softness and strength, relative to traditional paper based products were now available. This was mainly due to the use of long soft fibers, such as rayon and/or PET/PP in the spunlace process and the absence of binders in the fabric which were no longer required in a spunlace process.

In addition to these developments, recent innovations to wipe products include embossing or aperturing processes which are used primarily to improve the aesthetics of the wipe products.

For example, EP-A-705932 discloses non-woven fabrics having a planar background portion and at least one raised portion forming a three-dimensional pattern projected out of the plane of the background portion, wherein the base weight of the raised portion or portions is equal greater than the weight of the background portion.

EP-A-1032366 discloses personal cleansing wipes comprising a substrate from hydro-entangled fibers having a three-dimensional pattern wherein the density of the raised fibrous regions is the same as the density of the base surface, and an aqueous cleansing composition.

In addition to the above, lotions have been developed which offer skincare benefits in addition to the basic cleansing properties of the wipe. In addition, lotions have been introduced that are based on oil-in-water emulsions to deliver useful properties such as mildness, moisturization, protection and skin smoothness. Other aqueous based lotions have been developed to incorporate active skincare ingredients, such as chamomile, thereby delivering useful properties such as soothing. Current wet wipe products continue to use these approaches in that they are impregnated with either aqueous lotions or with oil in water emulsions.

This is in particularly useful in wipes intended for personal cleansing and in particular in wipes used for babies and infants. In the latter instance wet wipes are conventionally used for cleansing the perineal region when changing diapers. Inadequate cleaning not only results in personal discomfort but also gives rise to diaper rash and other infection related phenomena. Wipe products have become an important product category that has found a wide variety of applications for use in cleansing the perineal region when changing diapers. Inadequate cleaning not only results in personal discomfort but also gives rise to diaper rash and other infection related phenomena. It has been shown that the most effective way of preventing diaper rash is to cleanse the skin thoroughly and to remove the microorganisms that have been identified as causative. The source of these microorganisms is often the fecal deposits that can remain on a baby's skin while wearing the diaper.

Regardless of its end use the softness of the wipe product is of primary importance to the consumer. Softness of the wipe material on the one hand and perceived softness of the skin after usage of the wipe on the other hand are important consumer benefits. This in particular is the case for applications on babies.

U.S. Pat. No. 5,648,083 describes personal care compositions including silicones and polymeric emulsifier for providing barrier protection against dermatitis for baby wipes.

EP-A-705932 discloses non-woven fabrics having a planar background portion and at least one raised portion forming a three-dimensional pattern projected out of the plane of the background portion, wherein the base weight of the raised portion or portions is equal greater than the weight of the background portion.

EP-A-1032366 discloses personal cleansing wipes comprising a substrate from hydro-entangled fibers having a three-dimensional pattern wherein the density of the raised fibrous regions is the same as the density of the base surface, and an aqueous cleansing composition.

In addition to the above lotions have been developed which offered skincare benefits in addition to the basic cleansing properties of the wipe. One approach was the introduction of lotions that were based on oil-in-water emulsions which delivered useful properties such as superior mildness, moisturization, protection and skin smoothness, when compared to simple aqueous cleansing formulations.

W099/25318 discloses a personal cleansing wipe comprising, a single layer, nonwoven substrate formed from hydro-entangled fibers having a three-dimensional pattern and an aqueous liquid cleansing composition comprising an effective amount of a cleansing surfactant, the aqueous liquid cleansing composition being coated onto or impregnated into the substrate in particular proportions.

W099/13861 discloses a disposable, single use personal care cleansing and conditioning article comprising, inter alia, a water insoluble substrate wherein at least a first portion of said substrate is wet extensible and at least a second portion of said substrate is less wet extensible than said first portion and at least one lathering surfactant added onto or impregnated into the substrate, wherein said article is substantially dry prior to use.

### SUMMARY OF THE INVENTION

The present invention relates to a baby wipe product comprising a substrate and a mild cleansing liquid emulsion. The wipe is sized and configured and contains sufficient emulsion to enable a user to effectively cleanse an infant's entire body with a single wipe. The wipes are preferably made from nonwoven fabrics and may be in the form of a wet wipe or a dry wipe.

It is an object of this invention to provide products for head to toe cleansing of an infant that allows a care giver a convenient and quick application that is easy to carry.

### DETAILED DESCRIPTION OF THE INVENTION

The present invention is directed to a novel baby wipe that is adapted to act as a bath alternative for infants. The wipe is sized and configured to enable a user to clean a baby's entire body with a single wipe thereby obviating the need for a bath. In contrast to normal baby wipes, which are intended for cleansing only a small portion of a baby's body, such as the need to cleanse a portion of the baby's body during a diaper change, the wipes of the present invention have a sufficient size, thickness and volume of a mild cleansing liquid emulsion to enable total baby body cleansing.

The wipes of the invention comprise a substrate and a mild cleansing liquid emulsion that has been impregnated into the substrate. As used herein, the terminology “wipe” means a substrate and a liquid emulsion which are precombined for later use. The substrate comprises a porous material which is capable of holding a liquid emulsion within the pores of the substrate. Suitable substrates are known in the art of wipes and include, but are not limited to woven fabrics, knit fabrics, nonwoven fabrics, laminates of a fabric and a polymeric film, flocked fabrics, and combinations thereof.

Methods of making these substrates are also well known in the art and any of the conventional manufacturing methods are suitable for making the substrates of the present claimed invention.

### Substrates

In a preferred embodiment, the substrate is a non-woven fabric. One type of nonwoven fabric utilized in the present invention is made by either an air-laid or a wet-laid process in which fibers are first cut to desired lengths from long strands of filaments, passed into either water or an air stream, and then deposited onto a screen through which the fiber-laden air or water is passed. The deposited fibers are then bonded together to form the nonwoven fabric. The nonwoven fabric can be made from synthetic fibers or natural fibers. Commonly used synthetic fibers include fibers formed from polyethylene terephthalate, polyethylene, polypropylene, rayon, cellulose ester, polyvinyl derivatives, and polyamides, such as nylon 6, nylon 66, or polyesters, such as polyethylene terephthalate and polybutylene terephthalate, or combinations thereof. Commonly used natural fibers include wood pulp, cotton, wool, silk, jute, hemp, linen, sisal, or ramie fibers or combinations thereof.

In one embodiment of the invention, the synthetic fibers may be formed from thermoplastic fibers. In accordance with this embodiment, the substrate utilized in the present invention may be thermally bonded nonwoven fabric. In this process, the nonwoven fabric is formed by a spun bonding process, i.e., the fibers are spun out onto a surface and bonded by melting the fibers together by heat or chemical reactions. Typical thermoplastic fibers include polyesters, polyamides and polyolefins, such as for example, polypropylene, polyethylene, polyester, e.g. PET and PBT, and polylactide fibers or mixtures thereof.

Alternatively, the substrate may be formed by a spunlacing process. Fibers suitable for spunlacing include regenerated cellulose fibers such as rayon, viscose and/or lyocell or synthetic fibers such as polyester fibers, e.g. polyethylene terephthalate fibers (PET), or mixtures thereof.

In yet another embodiment the substrate can be formed from two, three, four or even more carded precursor webs which are at least partially superimposed onto each other to form a sandwich structure. The sandwich structure is subjected to a hydroentanglement procedure.

In accordance with this embodiment of the present invention, fibers are subjected to carding by use of at least one carding unit to produce a first precursor substrate layer. Onto this first precursor substrate layer at least one second fibrous precursor substrate layer is placed by airlaying. The second precursor substrate layer is optionally at least partially covered by a third precursor substrate layer comprising at least one third fibrous layer of non-woven fibers having been subjected to carding by use of at least one carding unit. Depending on the thickness desired, additional precursor substrate layers may be added utilizing the same procedure as above. The precursor substrate layers, when placed on top of each other, form the precursor substrate which is then subjected to hydroentanglement thus providing a composite system wherein all layers are tightly bond to each other to form the final substrate. In a particular execution, a first layer is formed by carding staple fibers, covering this layer with a second layer of pulp by airlaying and then covering the pulp layer with a carded layer of staple fibers. This three layer structure is subsequently entangled by a hydroentanglement process to form the final substrate. Preferably, the precursor sandwich structure is calendared. In accordance with this embodiment, it is preferred that the nonwoven substrate is derived from hydroentangling a multi-layer composite nonwoven, wherein the intermediate layer has been formed by airlaying. The precursor substrate comprises at least one first precursor substrate layer obtained by carding, followed by at least one second precursor substrate layer obtained by airlaying, followed by at least one third precursor substrate layer obtained by melt blowing, which in turn is followed by at least one fourth precursor substrate layer obtained by carding. These first to fourth precursor substrate layers when superimposed on top of each other form the precursor substrate which is then subjected to the hydroentanglement procedure web bonding step. For layers obtained by carding, it is preferred to use regenerated cellulose fibers, e.g. viscose, polypropylene, polyamide, polyester, e.g. polyethylene terephthalate, polylactide acid, polyvinyl alcohol fibers and/or natural fibers such as wood pulp, cotton, wool, silk, jute, hemp, linen, sisal, or ramie fibers or combinations thereof.

The wood pulp may be derived from softwood and/or hardwood and have fiber lengths of about 3 mm and 1 mm in length respectively. Since wood pulp fibers have a high surface area and a flat ribbon-like morphology, they provide the wipe with enhanced cleaning properties. Wood pulp fibers can also be sourced from bamboo pulp. These pulp fibers naturally have good absorbency and strength properties and since they are generally softer than other wood pulp fibers, are preferred in the present invention because they help prevent irritation to a baby's skin. When cotton fibers are used to form the substrate, the fiber length of cotton can vary from about 3 mm to about 30 mm. The use of cotton in the wipe enhances its cleaning properties due to the flat ribbon-like surface of the cotton fibers. The wipes of the present invention may either be single ply or multi-ply, e.g. two or more ply substrates laminated to each other by means well known in the art.

It is considered an important feature of the present invention that the wipes of the present invention have a size greater than 350 square centimeters and are preferably at least about 400 square centimeters, more preferably at least about 500 square centimeters and most preferably at least about 600 square centimeters. The shape of the wipe is not per se critical to the invention, and can be any commonly used shape as currently commercialized. A preferred shape is either square or rectangular to enable conventional roll goods processes to be used without excessive waste during manufacturing. In a preferred embodiment, the wipe is at least about 20 centimeters wide and at least about 30 centimeters long. Since the wipes of the present invention provide the user with the equivalent of a bath in every wipe, the size of the wipe must be sufficiently large to allow the baby caregiver the ability of cleansing a baby on non-bath days with a convenient no rinse required solution for a clean baby.

In an optional embodiment of the present invention, the porous absorbent substrate is a nonwoven sheet wherein a substantial portion of one or both surfaces of the sheet has a three-dimensional pattern embossed therein. The pattern is in the form of a series of raised and lowered regions. The raised region has a basis weight that is essentially the same as the basis weight of the lowered region; however the raised region has a density that is lower than the density of the lowered region.

The three dimensional pattern may be imparted to the porous absorbent substrate by providing a precursor substrate, providing a forming member having a surface with a three-dimensional pattern engraved therein which includes a series of raised and lowered regions. The shape of the three dimensional pattern is imparted to the substrate by advancing the precursor substrate onto the three-dimensional transfer device and applying hydraulic energy to the precursor substrate to impart the three dimensional pattern to the substrate.

In still further specific embodiments, the raised and lowered regions can be in the form of a pattern of lines or stripes. These lines preferably run in a single direction continuously from one side of the sheet to the other side. In a particular aspect, at least a section of one or both surfaces of the sheet has a ridged pattern. The ridged surface is comprised of a three dimensional pattern embossed in the substrate of the sheet, wherein the pattern comprises a repetitive series of raised and lowered regions in the shape of lines or stripes having equal or about equal width and having equal or about equal size. It is preferred that none of the raised or lowered regions has any flat portion and the basis weight of the substrate is substantially the same over the entire substrate. In this embodiment, the density of the raised portions is lower than the density of the lowered portions. In accordance with the foregoing embodiment of this invention, the raised and lowered regions are located adjacent to one another wherein a lowered region is followed by a raised region, followed by a lowered region, and so forth. In particular, the raised and lowered regions form one or zigzag pattern, or a pattern of multiple lines or stripes.

The depth and height of the lowered and respectively raised regions is generally in the range of 0.05 to 1 mm, preferably in the range from 0.1 to 0.7 mm, more preferably in the range of from 0.2 to 0.5 mm and most preferably from 0.2 to 0.4 mm, or about 0.3 mm.

The lines or stripes of raised and/or lowered regions can be continuous or discontinuous, either in a regular pattern or in a random pattern. If the lines or stripes have a wave-like pattern the amplitude of these waves and/or the length of these waves can be varied in broad ranges. Such alterations can take place on lowest point of the lowered regions may vary but preferably is in the range of 0.1 to 2 mm, more preferably from 0.2 to 1 mm, still more preferably from 0.3 to 0.7 mm, or about 0.5 mm. The thickness of the sheet at its thickest portion may equally vary and preferably is as the thickness of the sheet at its one or both sides of the absorbent sheet. Preferably, the density of the lowered regions is in the range of about 40 to 300, preferably of about 100 to 180 and most preferably of about 120 to 170 kg/m3, and the density of the raised regions is in the range of about 20 to 120, preferably 25 of about 50 to 100 and most preferably of about 60 to 90 kg/m3.

In a cross-sectional view of the absorbent sheet a pattern may be in the form of an alternating raised and lowered regions. For example, a pattern can be in a series of recurring lowered and raised regions in a symmetrical pattern of about the same size or alternatively, they may be unsymmetrical in shape. An unsymmetrical raised or lowered region can for example comprise two or more undulations in a recurrent pattern of raised and lowered regions that comprises a first raised region of larger height and a second and third raised regions being both of lower height whereas the width between the lowered regions of the first and second raised region is several times broader than the width between the lowered regions of the between consecutive raised regions. In preferred embodiments of this invention, the shape of the raised and lowered regions is substantially identical. As used herein, the term “substantially identical” means that the size or shape is essentially the same, with small difference of e.g. less than 10% or less than 5%. In still further specific embodiments, the raised and lowered regions can form a linear pattern that runs in one direction continuously from one side of the sheet to the other side.

It is important that the baby wipes of the present invention have a high wet strength of the substrate. This is achieved by the addition of suitable binding materials known in the art as wet strength resins. Increased wet strength can also be obtained by using spunlace or hydroentanglement techniques as previously disclosed herein. It is preferred to provide high wet strength by means of spunlacing and hydroentanglement due to the softness of the final nonwoven substrate. In a most preferred embodiment the nonwoven substrate is prepared by a hydroentanglement process.

The absorbing ability of the sheet material is important with regard to the applications envisaged by the present invention. During production the liquid emulsion is preferably taken up quickly by the substrate. In certain embodiments of this invention the wipes will be packaged in a stack containing a plurality of folded wipes. The absorbing ability of the substrate should be such that a chromatographic effect (sinking down of the lotion) in the stack is avoided during storage. However, it is important that during the usage of the wipe the impregnating solution is delivered evenly to the skin. The absorbing capacity of the sheet material is determined by the following parameters: the surface weight of the substrate, the nature of the raw materials used in the substrate, the manufacturing process used and the density of the substrate.

The selection of the raw materials of which the substrate is made depends on the manufacturing procedure. Typically in the manufacture of nonwoven substrates by the hydroentanglement process, use is made of mixtures of cellulose fibers and synthetic fibers. The relative quantity of synthetic fibers in the nonwoven substrate is from 0 to 100% and preferably is between 10 and 70%, more preferably in the range of 30 to 50% (all percentages being w/w). The porous, absorbent substrate of this invention can be made by a process that comprises placing a web of fibers on or against an appropriately shaped forming member having elevations and depressions. Depending on the production set up, the web can be placed on the forming member or vice versa the forming member can be placed on the web. The forming member a roll or drum or part of a roll or drum, e.g. the member can be curved and can be mounted on a roll or forming member under the influence of the pressure of the water jet of the hydrodynamic needling during the hydro entanglement procedure. In the latter instance, the forming member is perforated to allow the water of the water jets to become removed. A particular useful forming member in this instance is the above mentioned forming member of mesh wire. In a particularly preferred embodiment, the substrate, which preferably is formed using a spunlace process, using a hydrodynamic needle process wherein hydroentanglement takes place, and is then embossed to form raised and recessed regions.

### Wet Wipes

In a preferred embodiment, the baby wipe of the present invention is a wet wipe and the substrate is formed from a mixture of polyethylene terephthalate fibers and viscose fibers, wherein the polyethylene terephthalate fiber content ranges from about 50% to about 90% w/w, preferably from 75% to about 85% w/w and most preferably is about 80% w/w. Accordingly, the viscose fiber content would correspondingly range from 50% to 10% w/w, preferably 25% to 15% w/w and is most preferably about 20% w/w.

While the basis weight of the wet wipe substrate may vary somewhat, it is generally greater than about 30 grams per square meter, and generally ranges from about 40 grams per square meter to about 65 grams per square meter, preferably from about 40 grams per square meter to about 55 grams per square meter and most preferably about 50 grams per meter. It has been found that wipes having a basis weight in the above ranges, when combined with the above size range, provide an adequate volume to contain sufficient amount of liquid emulsion to adequately cleanse a baby's entire body and yet is not too thick so that it inhibits intimate cleansing. Wipes having the combination of the above size and basis weight are capable of holding between 10 to 15 grams of liquid emulsion, which has been found to be sufficient to adequately cleanse a baby's body. The fabrics are preferably soft textured and are bigger and preferably thicker than standard wipes to enable a user to gently cleanse baby's body all over. Accordingly, the wipes are moist and ready to use on the skin instantly, with no need to dampen the fabric or rinse the skin afterwards.

### Dry Wipes

In an alternative embodiment of the invention, the baby wipe is a dry wipe. In accordance with this embodiment, the dry wipe utilizes a substrate having a different range of fibers than the wet wipe because the substrate must be able to hold the lotions during the subsequent addition of water by the consumer. In a preferred dry wipe, the substrate is formed from a mixture of polyethylene terephthalate fibers and viscose fibers, wherein the polyethylene terephthalate fiber content ranges from about 20% to about 80% w/w, preferably from 30% to about 40% w/w and most preferably is about 35% w/w. Accordingly, the viscose fiber content would correspondingly range from 20% to 80% w/w, preferably 70% to 60% w/w and is most preferably about 65% w/w.

While the basis weight of the substrate in a dry wipe may vary somewhat, it is generally greater than about 30 grams per square meter, and generally ranges from about 50 grams per square meter to about 140 grams per square meter, preferably from about 80 grams per square meter to about 100 grams per square meter and most preferably about 90 grams per meter. It has been found that wipes having a basis weight in the above ranges, when combined with the above size range, provide an adequate volume to contain sufficient amount of liquid emulsion to adequately cleanse a baby's entire body and yet is not too thick so that it inhibits intimate cleansing.

### Emulsions

The liquid emulsion added the wipe is in the form of a mild cleansing liquid that is formulated to gently cleanse a baby's sensitive skin without the need for water or rinsing. Accordingly, the emulsions of the present invention are substantially soap-free and utilize a non-drying/moisturizing formula wherein the emulsion contains a skin conditioning ingredient and/or emollient and/or humectant. In accordance with this embodiment, a user of the wipes of the present invention is advantageously not required to rinse the infant subsequent to providing a thorough cleansing with the wipe. In another preferred embodiment, the emulsion is free of dyes and is hypoallergenic. The liquid emulsions in their simplest form contain one or more surfactants, a humectant, water, a rheology modifier and a preservative. As used herein, compositions that are “mild cleansing liquid emulsions” (hereinafter “emulsion(s)” or “liquid emulsion(s)”) refer to compositions that have low skin irritancy properties and are mild to the eyes. Low skin irritancy is indicated by: a) a relatively high TEP value as determined in accordance with the TEP Test as set forth herein; and/or b) a passing score in the four screening tests (cell viability; cell lysis; and cytokine release (IL-1∝(and IL-1ra) performed in accordance with the Skin Assay Test as set forth herein. “Mild to the eyes” refers to compositions that possess a relatively high TEP value as determined in accordance with the TEP Test as set forth herein. Preferred mild cleansing emulsions are also substantially free of ocular sting. As used herein, a composition that is “substantially free of ocular sting” or “substantial lack of ocular sting” refers to compositions that possess relatively low sting values as determined in accordance with the Ocular Sting Test as set forth herein.

1) Skin Assay Test—Mildness is determined using a skin equivalent model as described by Bernhofer, et al., Toxicology in Vitro, 219-229 (1999), which is incorporated by reference herein. This model utilizes sequential screens for determining cell viability, cell lysis and cytokine release in order to evaluate the mildness of a surfactant system to the skin. Cell viability is determined using an alamarBlue® assay, which is an indicator of metabolic activity. Cell lysis is detected by measuring lactate dehydrogenase (LDH) activity released from the cytosol of damaged cells. Cytokine release (both IL-1∝ and IL-1ra) is measured for those sample sets which do not exhibit loss of cell viability or cell lysis.
In general, a EpiDerm® Epi-100 human epidermal model is obtained from MatTek Corporation (Ashland, Mass. USA) and maintained according to the manufacturers' instructions. Normal human-derived epidermal keratinocytes (NREK) are then cultured to form a multilayered differentiated model of the epidermis. After a set of NREKs is exposed in triplicate to 100 μl of a topically applied surfactant sample, it is incubated for about 1 hour. After incubation, the set is washed five times, 400 μl per wash, with phosphate buffered saline (PBS), placed onto a fresh assay media, and returned to the incubator for about 24 hours.
Cell viability of the NREKs is determined 24 and 48 hours post treatment with the alamarBlue® assay (Alamar Biosciences. Sacramento. Calif. USA) in accordance with manufacturers' protocols and a Cytofluor II Fluorescent Plate Reader (PerSeptive Biosystems. Framingham. Mass. USA). Cell lysis is determined colorimetrically using an LDH cytotoxicity detection kit (Boehringer-Mannheim). Cytokine content is measured using human calorimetric ELISA kits for IL-1∝ (ENDOGEN. Cambridge, Mass. USA), interleukin-1 receptor antagonist (IL-1ra, R&D Systems. Minneapolis. Minn. USA), granulocyte⁻macrophage colony stimulating factor (GM-CSF). interleukin-6 (IL-6), interleukin-8 (IL-8). interleukin-I0 (IL-b) and TNF∝ (PerSeptive Diagnostics. Cambridge, Mass. USA).
2. Ocular Sting Test: Using a double-blinded, randomized, two (2) cell study test design, one (1) drop of a sample (e.g. a 10% dilution of a cleansing composition in water) at a temperature of about 38° C. is instilled into a subject's eye. A new sterile disposable eyedropper is used for each sample and disposed of after being used on only one individual's eye. All instillations are performed either by an investigator or by a trained technician.
Within 30 seconds, or as closely as possible following instillation, the subject is asked to grade the perceived stinging sensation to the eye utilizing the following criteria:
∘Sting
∘0=Within normal limits
∘1=Mild, very slight
∘2=Moderate
∘3=Severe
   After 15 minutes and 60 minutes post-instillation, the subject is again asked to grade the perceived stinging sensation to the eye.
      3.) Trans-Epithelial Permeability Test (“TEP Test”): Irritation to the eyes expected for a given formulation is measured in accordance with the Invittox Protocol Number 86, the “Trans-epithelial Permeability (TEP) Assay” as set forth in Invittox Protocol Number 86 (May 1994). In general, the ocular irritation potential of a product can be evaluated by determining its effect on the permeability of a cell layer, as assessed by the leakage of fluorescein through the layer. Monolayers of Madin-Darby canine kidney (MDCK) cells are grown to confluence on microporous inserts in a 24-well plate containing medium or assay buffer in the lower wells. The irritation potential of a product is evaluated by measuring the damage to the permeability barrier in the cell monolayer following a 15 minute exposure to dilutions of the product. Barrier damage is assessed by the amount of sodium fluorescein that has leaked through to the lower well after 30 minutes, as determined spectrophotometrically. The fluorescein leakage is plotted against the concentration of test material to determine the EC50 (the concentration of test material that causes 50% of maximum dye leakage, i. e., 50% damage to the permeability barrier). Higher scores are indicative of milder formulas.

Exposure of a layer of MDCK cells grown on a microporous membrane to a test sample is a model for the first event that occurs when an irritant comes in contact with the eye. In vivo, the outermost layers of the corneal epithelium form a selectively permeable barrier due to the presence of tight junctions between cells. On exposure to an irritant, the tight junctions separate, thereby removing the permeability barrier. Fluid is imbibed to the underlying layers of epithelium and to the stroma, causing the collagen lamellae to separate, resulting in opacity. The TEP assay measures the effect of an irritant on the breakdown of tight junctions between cells in a layer of MDCK cells grown on a microporous insert. Damage is evaluated spectrophotometrically, by measuring the amount of marker dye (sodium fluorescein) that leaks through the cell layer and microporous membrane to the lower well.

The liquid emulsions of the present invention have a TEP value greater than one, preferably greater than 2 and most preferably greater than 2.1. Alternatively, or in addition to the foregoing TEP values, the liquid emulsions of the present invention have an Ocular Sting value of less than 3, preferably less than 2 and most preferably less than 1.

### Surfactants/Emulsifiers

Suitable surfactants for use in this invention include nonionic surfactants, amphoteric surfactants, anionic surfactants or mixtures thereof. Examples of non-ionic surfactants include PEG-6 caprylic/capric glycerides (available from Croda, Inc., Parsippany, N.J.), polysorbate 20, and PEG-80 sorbitan laurate (available from Uniqema, Wilmington Del.). Examples of amphoteric surfactants include but are not limited to alkylimino-diproprionates, alkylamphoglycinates (mono or di), alkylamphoproprionates (mono or di) alkylamphoacetates (mono or di), N-alkyl P-aminoproprionic acids, alkylpolyamino carboxylates and phosphorylated imidazolines. The preferred amphoteric surfactants are disodium lauroamphodiacetate, sodium lauroampho PG-acetate, sodium cocoamphoacetate, and disodium cocamphodipropionate. The particularly preferred amphoteric surfactants are disodium lauroamphodiacetate. One commercial supplier of this material is Mona Industries under the trade name Monateric 949-J. The nonionic surfactants include the fatty alcohol acid or amide ethoxylates, monoglyceride ethoxylates, sorbitan ester ethoxylates, and alkyl polyglycosides. When using an amphoteric surfactant, the amount, based upon the total weight of the solution, is from about 0.01% to about 10%, preferably from about 0.05% to 5%, and more preferably, from about 0.10% to about 0.5%. When using a nonionic surfactant, the amount is typically, from about 0.02% to about 15%, preferably, from about 0.10% to about 10%, and more preferably from about 0.25% to about 2%.

The liquid component of the wipes of the present invention is preferably emulsion-based. The emulsions can be oil-in-water or water-in-oil emulsions, or be of more complex nature such as water-in-oil-in-water. Preferred emulsions are oil-in-water emulsions.

The products of the present invention may further contain zwitterionic, amphoteric, cationic and or anionic surfactants that may also function as emulsifiers. Zwitterionic surfactants are those surface active compounds, that contain at least a quaternary ammonium group and at least a —COOH or -S03 C-1 group. Particularly alkyl sulfoacetates, e.g. sodium lauryl sulfoacetate; acyl isothionates, alkyl ether sulfosuccinates, e.g. disodium laureth sulfosuccinate, acyl sarcosinates, acyl taurines with linear alkyl- or acyl groups having 12 to 18 C-atoms as well as alkali- or ammonium salts of sulfosuccinates and acyl glutamates. Quaternary ammonium derivatives can in particular be used as cationic surfactants. Preferred are ammonium halo- genides, in particular chlorides and bromides, e.g. alkyl tri-methylmonium chloride, dialkyl dimethylmonium chloride and trialkyl methylammonium chloride, z. B. cetyl trimethylammonium chloride, stearyl trimethylmonium chloride, distearyl dimethylammonium chloride, Iauryl dimethylammonium chloride, Iauryldimethylbenzylammonium chloride and tricetyl methylmonium chloride. Additional cationic surfactants are the quaternary esters with good biological degradability, such as, for example, dialkylammo-nium methosulfates and methylhydroxyalkyl dialkoyloxy alkylammonium methosulfates (sold under the tradename Stepantex® and the products of the Dehyquart®-series). Further cationic surfactants are the quaternized protein hydrolysates. Non-ionic surfactants comprise alkyl gluco-sides, e.g. decyl glucoside, laurylglucoside; alkyl isothionates; Suitable conditioners are e.g. alkylamido ammonium lactate, cetrimonium chloride and distearoylethyl hydroxyethyl-useful zwitterionic surfactants are the so-called betaines such as N-alkyl-N,N-dimethyl ammonium glycinate, for example coco-alkyl dimethylammonium glycinate, N-acyl-aminopromonium methosulfate and cetearyl alcohol, cetyl dimethi-cone, cetyl ricinoleate, dimethicone, laureth-23, laureth-4, polydecene, retinyl palmitate, agents selected from glyceryl monooleate and cocoglucoside including mixtures thereof (in particular the product ‘Lamesoft®’ of Cognis which is a mixture of these two components), quaternized protein hydrolysates, quaternized cellulose and starch derivatives, quaternized copolymers of acrylic or methacrylic acid or salts, quaternized silicone derivatives, silicone oils, cyclom-ethicones, and the like agents, including mixtures thereof.

Preferred are nonionic emulsifiers/surfactants, which typically have good skin compatibility. Improved sensory properties are obtained when combining non-ionic W/O and O/W emulsifiers. The compositions may contain the emulsifier(s) in an amount of from zero to about 20% (w/w), respectively 0.1 to 15% (w/w) and in particular 0.1 to 10% (w/w) relative to the total quantity of the compositions. The emulsions in the products of the invention may further contain one or more emulsifiers/surfactants that are either nonioninc, anionic, amphoteric or a combination thereof resulting in a composition that is mild to the eyes and skin. Emulsifiers are generally present in an amount that is in the range of 1 to 25%, in particular 5 to 20% and more in particular 5 to 15%. Because fecal deposits consist of both water-soluble and oil-soluble matter, complete removal of fecal deposits from the diaper area requires both water-based and oil-based surfactants, or combinations thereof, that provide effective cleansing.

In a preferred embodiment, the liquid emulsion contains the following components: a thickening agent such as a carbomer, a pH adjuster such as sodium hydroxide and or citric acid, a moisturizing agent such as glycerin, and a cleansing agent such as coco-glucoside, glyceryl oleate, lauryl glucoside, polyglyceryl-2 dipolyhydroxystearate, glyceryl polyacrylate, and a preservative such as p-anisic acid, phenoxyethanol and/or sodium benzoate, and/or a rheology modifier such as potassium acrylates copolymer, superhydrophilic amphiphilic copolymer as described in publication US 2011/0081309 A1 and/or polyglyceryl 10-Laurate. Baby wipes typically are soft and are loaded with a cleanser and moisturizer.

Suitable thickeners are e.g. acrylates/steareth-meth-acrylate copolymer, acrylates/C10-30 alkyl acrylate crosspolymer, carbomer, carboxymethyl starch, cera alba, cetyl hydroxyethylcellulose, dimethicone/vinyl dimethicone crosspolymer, propylene glycol alginate, hydroxyethylcellulose, hydroxypropyl methylcellulose, silica, silica dimethyl silylate, xanthan gum, hydrogenated butylene/ethylene/styrene copolymer. The quantity of thickener can vary but usually is in the range of about 0.01 to 2% (w/w).

In a preferred embodiment, the liquid emulsion contains one or more anti-microbial agents. Examples of suitable anti-microbial agents are those active against gram-positive bacteria such as, glycerin monolaurate, glycerin stearate, glycerin-oleate and glycerine-dioleate, phenethyl alcohol, phenoxyethanol, quaternary ammonium compounds. Examples of such compounds include but are not limited to benzalkonium chloride, benzethonium chloride, cetylpyridinium chloride and the like, and mixtures thereof. The preferred cationic antibacterial agent is benzalkonium chloride. The quantity of anti-microbial agents can vary but usually is in the range of about 0.01 to 2% (w/w) relative to the total amount of the emulsion. The liquid emulsion may also contain anti-microbial agents active against yeast and mold growth. Examples of such ingredients are organic acids, such as sodium benzoate and potassium Sorbate as well as methylparaben, propylparaben and quaternium-15. Preferably, the anti-microbial agent is present in the liquid emulsion at a concentration (weight/weight) of from about 0.05% to about 0.5%, preferably from about 0.15% to about 0.50%, and more preferably from about 0.2% to about 0.30%. The presence of these anti-microbial agents is particularly useful in products that are applied on babies because of their mildness and lack of side effects.

The emulsions of the present invention further comprise at least one emulsion stabilizer. Examples of suitable emulsion stabilizers include small chain nonionic surfactants, such as polymeric ethers, including, for example, polyoxyethylene-polyoxypropylene block polymers and nonionic surfactant consisting of a mixture of laurate esters of sorbitol and sorbitol anhydrides, condensed with approximately ethylene oxide. Particularly preferred nonionic surfactants useful as emulsion stabilizers include, for example Poloxamer 124 which is a non-ionic surfactant belonging to the chemical class of polymeric ethers, consisting of a polyoxyethylene, polyoxypropylene block polymer and polysorbate 20, a nonionic surfactant consisting of a mixture of laurate esters of sorbitol and sorbitol anhydrides, condensed with approximately 20 moles of ethylene oxide.

The inclusion of small chain nonionic surfactants at low levels stabilizes the polymeric emulsifier at a pH less than 7.0 for incorporation into fabric. In one embodiment, the pH of the emulsion of the invention can range from about 4.0 to about 5.8. The pH can be adjusted by adding one or more pH-adjusting agents in an amount effective to provide such pH values. Agents that may be used to adjust the pH of the compositions herein include organic and inorganic acids and bases, such as sodium hydroxide and/or citric acid, sufficient to provide a pH range of about 4.0 to about 7.0. An example of a suitable base is sodium hydroxide. The viscosity of the liquid emulsion is preferably less than 1000 cps which allows for impregnation into fabric in the conventional manner.

The emulsion can further contain one or more moisturizers. These are added to improve the sensory properties as well as to regulate skin hydration. These agents additionally can improve the penetration of the emulsion into the substrate. The emulsions used in the wipe product according to the invention are mild to the skin and aesethically pleasing due to the absence of alcohols, such as ethanol. However, the wipe product preferably contains a skin moistener, humectant, or emollient such as a water-soluble or water miscible polyol. Suitable water-soluble polyols include water-soluble alkylene polyols and water-soluble analogs of such polyols. Water-soluble analogs of these polyols include water-soluble esters of alkylene polyols. Non-limiting examples of water-soluble polyols suitable for use herein include ethylene glycol, propylene glycol, butylene glycol, diethylene glycols, triethylene glycols, other water-soluble polyethylene glycols, water-soluble soluble polypropylene glycols, hexylene glycol, glycerol, polyoxyethylene sorbitol, 1,2,4-butanetriol, 1,2,6-hexanetriol, sorbitol and he like and mixtures thereof. Generally, the polyol or humectant is present in amounts ranging from about 0.5% to about 20% by weight, preferably from about 0.5% to about 5% by weight, most preferably from about 0.5% to about 2% by weight of the composition. The compositions of the invention can comprise less than 5% by weight of a polyol and are preferably free of ethanol. The emollients that may be added comprise lipids like lanolin, lanolin alcohols, lanolin acids, polyethoxylated or acylated lanolin or lanolin derivatives, lecithin and lecithin derivatives, fatty alcohols, either linear or branched with chain lengths between C6 and C40, and their esters with organic acids, e.g. carbonic acids or polyacids containing between 2 and 30 C atoms, branched, aromatic or linear including hydroxy or amino acids, fatty acids and fatty acid esters with alcohols or poly alcohols containing between 2 and 40 C atoms, branched, aromatic or linear, sterols found in the unsaponifiable fraction of e.g. avocado oil, almond oil, soybean oil, etc. like soy phytosterol, -sitosterol, -sitosteryl laurate, -sitosteryl stearate, etc. natural and synthetic waxes, e.g. bees wax, purcelline, shea butter, cocoa butter, ceresin, petrolatum, micro wax, camauba wax candelilla wax and alike. Silicones, such as dimethicone, may also be used. Suitable moisturizers are amino acids, pyrrolidone carbonic acid, lactic acid and its salts, lactitol, urea and urea 25 derivatives, ureic acid, glucosamine, creatinine, hydrolysis products of collagen, chitosan or chitosan salts/-derivatives, and in particular polyols and polyol derivatives (e.g. ethylene glycol, propylene glycol, butylene glycol, pentylene glycol, hexylene glycol, erythrite, 1,2,6-hexanetriol, polyethylene 30 glycols such as PEG-4, PEG-6, PEG-7, PEG-8, PEG-9, PEG-10, PEG-12, PEG-14, PEG-16, PEG-18, PEG-20, PEG-135, PEG 150), sugar and sugar derivatives (e.g. fructose, glucose, maltose, maltitol, mannite, inosite, sorbite, sorbityl silandiol, sucrose, trehalose, xylose, xylit, glucuronic acid and its salts), ethoxylated sorbitol (Sorbeth-6, Sorbeth-20, Sorbeth-30, Sorbeth-40), honey and hydrogenated honey, hydrogenated starch hydrolysates, as well as mixtures of hydrogenated wheat protein, hydrolyzed milk protein, lecithin, pythantriol, hyaluronic acid and salts thereof, and PEG-20-acetate 40 copolymers. Particularly preferred moisturizers are glycerin, glyceryl oleate, polyglyceryl 10-laurate, di-glycerin and tri-glycerin.

Another optional component useful in the emulsions according to the invention is any of the anti-irritants known in the art. The anti-irritant should be present in an amount effective to provide anti-irritation properties to the compositions. An example of a suitable anti-irritant is allantoin which is generally present at from about 0.05 to about 4.00% by weight, more preferably, from about 0.05 to about 0.5% by weight.

The baby wipe products according to the invention may also contain sunscreen filters. In accordance with this embodiment, the emulsion contains sunscreen filters which can include organic compounds and or inorganic filters which are capable of absorbing ultraviolet radiation. It is preferred that the sunscreen filters are capable of absorbing both UVA and UVB radiation.

It should be noted that all of the foregoing compositions and ranges are set forth for wet wipe applications. In a dry wipe application, upon evaporation of water from the wipe, the final ranges of excipients in the packaged product would increase by a factor of about 40-80%.

### Application of the Emulsions

The liquid emulsions may be applied to the substrate using methods generally known in the art for applying aqueous or nonaqueous compositions. For liquid emulsions that are not too viscous, techniques such as spraying, dripping, immersing and the like can be applied. They also can be applied at the surface or at the surface portion of the sheet, on one or on both sides. The emulsions can be applied evenly or non-evenly to the sheet, non-evenly meaning that the distribution of the amount of the composition varies over the area of the sheet, i.e. some areas of the sheet can have greater or lesser amounts of the composition. Preferably the composition is evenly applied to the area of the sheet. The compositions can be applied discontinuously or continuously to one or both sides of the sheet, or it may even be applied as a complete covering of one or both surfaces of the wipe.

The compositions may be applied in various ways, evenly or non-evenly, continuously or non-continuously, at the surface or surface portion or, preferably, throughout the whole of the sheet material. Compositions may be applied at both sides or only at one side of the sheet.

The wipes of the invention are preferably combined, e.g., coated or impregnated, with the liquid emulsions by dipping the wipes in the liquid emulsion, spraying the liquid emulsion onto the wipes, and other means known in the art. A preferred application method for the emulsion is by spraying with a suitable nozzle or by dripping, for example by using a perforated tube having holes or slits. The immersing technique can be done by running the substrate through a bath holding the emulsion and subsequently controlling the amount of liquid that is absorbed by pressing. The amount of such emulsion on the wipe may be in the range from about 100 to about 400%, preferably from about 200% to about 400%, expressed as the weight of the composition relative to the weight of the sheet in dry condition.

In a particular execution, the sheet is cut into strips the transverse size of which being similar to the size of the end wipe. Subsequently the carrier strips are folded according to methods generally known and applied in the art. In a further step, the strips are cut so that the desired size of the sheets, in particular of the wipes, is obtained. The thus obtained wipes can be packed individually or can be stacked in a predetermined number, generally between 10 and 30 wipes per package, preferably between 15 and 25, most preferably about 20. The stack is packed in a suitable packaging container, for example a plastic wrap or box. When removed from its container, the wet wipes of the present invention are not only sufficiently wet, and thus do not require further wetting, but also possess a soft feel to the touch.

What is claimed is:

1. A wet wipe product comprising a substrate and a stable emulsion, wherein the wipe is sized and configured to enable a user to clean a baby's entire body thereby obviating the need for a bath, wherein the substrate has a size greater than 350 square centimeters and wherein the liquid emulsion has a TEP value greater than one.

2. The wet wipe according to claim 1, wherein the substrate has a size at least about 600 square centimeters.

3. The wet wipe according to claim 1, wherein the substrate is a non-woven fabric and the emulsion is evenly dispersed throughout the wipe.

4. The wet wipe according to claim 1, wherein the emulsion has an Ocular Sting value of less than 3.

5. The wet wipe according to claim 4, wherein the emulsion has an Ocular Sting value of less than 2.

6. The wet wipe according to claim 4, wherein the emulsion has an Ocular Sting value of less than 1.

7. The wet wipe according to claim 1 wherein the substrate is formed by a spunlacing process.

8. The wet wipe according to claim 1 wherein the substrate is a porous absorbent nonwoven sheet wherein a substantial portion of one or both surfaces of the sheet has a three-dimensional pattern embossed therein, wherein the pattern is in the form of a series of raised regions and lowered regions and the raised region has a basis weight that is essentially the same as the basis weight of the lowered region and the raised region has a density that is lower than the density of the lowered region.

9. The wet wipe according to claim 1 wherein the substrate is formed from a mixture of polyethylene terephthalate fibers and viscose fibers, wherein the polyethylene terephthalate fiber content ranges from about 50% to about 90% w/w.

10. The wet wipe according to claim 1 wherein the basis weight of the substrate is greater than about 30 grams per square meter.

11. A dry wipe product comprising a substrate and a liquid emulsion, wherein the wipe is sized and configured to enable a user to clean a baby's entire body thereby obviating the need for a bath, wherein the substrate has a size greater than 350 square centimeters and wherein the liquid emulsion has, a TEP value of greater than 2.

12. A dry wipe product comprising a substrate and a stable emulsion, wherein the wipe is sized and configured to enable a user to clean a baby's entire body thereby obviating the need for a bath, wherein the substrate has a size greater than 350 square centimeters and wherein the liquid emulsion has a TEP value of greater than 2.1.

13. A dry wipe product comprising a substrate and a stable emulsion, wherein the wipe is sized and configured to enable a user to clean a baby's entire body thereby obviating the need for a bath, wherein the substrate has a size greater than 400 square centimeters and wherein the liquid emulsion has a TEP value greater than one.

14. The dry wipe according to claim 12, wherein the substrate has a size at least about 600 square centimeters.

15. The dry wipe according to claim 12, wherein the substrate is a non-woven fabric and the emulsion is evenly dispersed throughout the wipe.

16. The dry wipe according to claim 15, wherein the emulsion has an Ocular Sting value of less than 3.

17. The dry wipe according to claim 15, wherein the emulsion has an Ocular Sting value of less than 2.

18. The dry wipe according to claim 15, wherein the emulsion has an Ocular Sting value of less than 1.

19. The dry wipe according to claim 12 wherein the substrate is formed by a spunlacing process.

20. The dry wipe according to claim 12 wherein the substrate is a porous absorbent nonwoven sheet wherein a substantial portion of one or both surfaces of the sheet has a three-dimensional pattern embossed therein, wherein the pattern is in the form of a series of raised regions and lowered regions and the raised region has a basis weight that is essentially the same as the basis weight of the lowered region and the raised region has a density that is lower than the density of the lowered region.

21. The dry wipe according to claim 12 wherein the substrate is formed from a mixture of polyethylene terephthalate fibers and viscose fibers, wherein the polyethylene terephthalate fiber content ranges from about 20% to about 80%.

22. The dry wipe according to claim 12 wherein the basis weight of the substrate is greater than about 30 grams per square meter.
